(19) United States
(12) Patent Application Publication
(10) Pub. No.: US 2024/0342080 A1
(43) Pub. Date: Oct. 17, 2024

(54) COMPOSITIONS AND METHODS FOR INHIBITING HUMAN BLOOD PROTEIN VITRONECTIN

(71) Applicant: Sanford Burnham Prebys Medical Discovery Institute, La Jolla, CA (US)

(72) Inventors: Francesca M. MARASSI, La Jolla, CA (US); Kyungsoo SHIN, La Jolla, CA (US); James E. KENT, La Jolla, CA (US)

(21) Appl. No.: 18/290,749

(22) PCT Filed: Jul. 20, 2022
(86) PCT No.: PCT/US2022/037704

§ 371 Date: Jan. 19, 2024

Related U.S. Application Data
(60) Provisional application No. 63/224,214, filed on Jul. 21, 2021.

(51) Int. Cl. A61K 9/00; C07K 16/18

(52) U.S. Cl. CPC A61K 9/0048; C07K 16/18

(57) ABSTRACT

A method for inhibiting activity of a human blood protein vitronectin is disclosed. The method includes administering a composition that inhibits activity of a calcium and hydroxyapatite binding site of the human blood protein vitronectin. An ophthalmic composition is also disclosed. The composition includes: an effective amount of an organic compound having a molecular weight of less than 1,000 Daltons; and one or more selected from the group consisting of a thickening agent, a pH adjustor, a wetting agent, a stabilizer, a solubilize, a preservative, a refreshing agent, and an ointment base.

### CROSS REFERENCE TO RELATED APPLICATIONS

This application claims the benefit of U.S. Provisional No. 63/224,214, filed on Jul. 21, 2021, which is incorporated by reference herein, in its entirety, for all purposes.

### GOVERNMENT RIGHTS

This invention was made with government support under GM118186 awarded by National Institutes of Health. The government has certain rights in the invention.

### FIELD OF THE INVENTION

The present invention relates to compositions and methods for inhibiting human blood protein vitronectin (Vn).

### BACKGROUND OF THE INVENTION

Geographic atrophy is the chronic progressive degeneration of macula, an area in the center of retina, as a part of late-stage age-related macular degeneration (AMD). The disease is associated by localized sharply demarcated atrophy of outer retinal tissue, retinal pigment epithelium and choriocapillaris. It starts typically in the perifoveal region and expands to involve the fovea with time, leading to central scotomas and permanent loss of visual acuity.

It is estimated that the number of people with AMD exceeds 196 million in 2020 and that number is expected to rise to 288 million by 2040. The indicator of progression to AMD includes the appearance of drusen, pebble-like calcified yellow-white protein-lipid deposits under the retina. There are two forms of AMD: exudative (wet) and non-exudative (dry). While there are currently some promising treatments for wet AMD, no FDA-approved treatment exists for dry AMD or geographic atrophy. There is a need for effective treatment for dry AMD or geographic atrophy.

### SUMMARY OF THE INVENTION

In one embodiment, the present invention provides a method for inhibiting activity of a human blood protein vitronectin. The method includes administering a composition that inhibits activity of a calcium and hydroxyapatite binding site of the human blood protein vitronectin. In some aspects, the composition inhibits AMD related drusenoid formation. In some aspects, the method reduces amount of ectopic deposits that are associated with AMD. In some aspects, the method prevents the formation of ectopic deposits that are associated with AMD. In some aspects, the method includes identifying a patient that is in need of a treatment to reduce the amount of ectopic deposits that are associated with AMD. In some aspects, the human blood protein vitronectin is presented in a human eye.

In another embodiment, the composition comprises an effective amount of an organic compound having a molecular weight of less than 1,000 Daltons.

In another embodiment, the composition is an ophthalmic composition.

In another embodiment, the composition further comprises one or more selected from the group consisting of a thickening agent, a pH adjustor, a wetting agent, a stabilizer, a solubilize, a preservative, a refreshing agent, and an ointment base.

In another embodiment, the thickening agent is gellan gum or xanthan gum.

In another embodiment, the calcium and hydroxyapatite binding site is an HX domain of the human blood protein vitronectin.

In another embodiment, the organic compound binds to the HX domain.

In another embodiment, the method is for treating geographic atrophy or age-related macular degeneration.

In another embodiment, the present invention provides an ophthalmic composition that includes an effective amount of an organic compound having a molecular weight of less than 1,000 Daltons; and one or more selected from the group consisting of a thickening agent, a pH adjustor, a wetting agent, a stabilizer, a solubilize, a preservative, a refreshing agent, and an ointment base.

In another embodiment, the thickening agent is gellan gum or xanthan gum.

In another embodiment, the pH adjustor is selected from the group consisting of hydrochloric acid, citric acid, phosphoric acid, acetic acid, sodium hydroxide, potassium hydroxide, boric acid, borax, sodium carbonate, sodium hydrogencarbonate and tris(hydroxymethyl)aminomethane.

In another embodiment, the wetting agent is selected from the group consisting of glycerin, carboxy methylcellulose, hydroxypropyl methylcellulose, mannitol, polyvinyl alcohol (PVA), and hydroxyethylcellulose.

In another embodiment, the ophthalmic composition is an ophthalmic solution or an ophthalmic ointment.

In another embodiment, the ophthalmic composition is for treating geographic atrophy or age-related macular degeneration.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 shows the fluorescence emission in an in vitro drusen model that shows that Vn promotes HAP formation

FIG. 2 shows the fluorescence emission in the in vitro drusen model that shows that Vn antibody inhibits Vn-orchestrated HAP formation.

### DETAILED DESCRIPTION OF THE ILLUSTRATED EMBODIMENTS

Reference will now be made in detail to embodiments of the present invention.

The extracellular deposits that accumulate under the retinal pigment epithelium of the aging eye are a hallmark of age-related macular degeneration. The ectopic deposits are rich in blood proteins, lipids, and hydroxy apatite. Calcified deposits have also been linked with progression of macular degeneration.

The human blood protein vitronectin (Vn) is human blood protein that interacts with multiple ligands to regulate hemostasis, cell adhesion and migration, innate immunity, tissue remodeling, and bone remodeling. Vn binds both soluble calcium ions and solid hydroxyapatite with chemical specificity. Vn may also nucleate biomineralization and aid drusenoid deposition around lipid droplets. Interfering with Vn/lipid/hydroxyapatite (“HAP”) spherules may disrupt AMD drusenoid formation. Accordingly, compositions that inhibit the activity of the calcium and HAP binding site of Vn may reduce or prevent the formation of ectopic deposits in the human eye that are associated with AMD.

In blood, Vn circulates as an intact 75,000 Da glycosylated molecule, or as two disulfide-linked 65,000 Da and 10,000 Da polypeptides. The Vn sequence begins with a 44-residue somatomedin B domain that is responsible for regulating plasminogen activation, followed by an ArgGly Asp motif that mediates binding to integrin receptors. These are linked to an HX domain by a 90-residue segment with predicted conformational disorder. The 325-residue HX domain includes about 70% of the sequence of mature Vn and contains important binding sites.

The structure of the HX domain includes a four-bladed β-propeller, with each blade formed by one βββα HX repeat and the termini connected by a disulfide bond. The propeller top (defined as the start of each β1) forms a smooth surface, while longer flexible loops protrude from the bottom. The four β1 strands meet at the propeller center to form a channel that occludes a metal-chloride-metal ion triplet. Inside the channel, chloride is bound by four β1 amide hydrogens, and each metal ion is coordinated by four BI carbonyl oxygens plus an oxygen from water or sulfate.

The HX domain of Vn is capable of binding both soluble ionic calcium and crystalline hydroxyapatite with high affinity and chemical specificity. Circulating Vn is calcium-bound in vivo. The calcium binding site maps to the top of the Vn-HX propeller, where four Asp generate a highly focused electronegative potential above the channel opening. Calcium is unlikely to be occluded inside the channel. The same site is involved in binding both ionic calcium and hydroxyapatite, and ionic calcium cooperatively enhances the affinity of Vn for hydroxyapatite.

The affinity of phospholipids for calcium is well known, and phospholipids have been shown to nucleate calcium-phosphate clusters on membrane surfaces. Lipid phosphate groups are thus expected to provide a template for Vn-mediated epitaxial mineralization of HAP on the surface of lipid droplets. The calcium binding affinity of Vn is sufficiently high to maintain circulating Vn in a calcium-bound state, yet sufficiently low for exchange of Vn-bound calcium with the surface of HAP or lipid droplets. Such calcium exchange interactions may thus promote the accumulation of a Vn surface layer that regulates HAP crystal growth and stabilizes it against dissolution. As such, compositions that inhibit the activity of the calcium and hydroxyapatite binding site of Vn may disrupt the formation of and/or destabilize and help reduce drusenoids and/or other ectopic deposits in the human eye that are associated with AMD.

The propeller structure of the major domain of Vn clasps free calcium and HAP calcium. Vn plays an active role in drusen formation. There are many HAP-binding proteins but Vn is unique in promoting HAP mineralization and deposition on lipids. This leads to understand how Vn orchestrates the. mineralization of HAP, which defines the bone-like shell of calcified drusen. Specifically, Vn initiates HAP formation by nucleating calciumphosphate clustering. The Vn propeller domain regulates exchange of soluble ionic calcium and phosphate with circulating lipids or the surface of HAP.

An in vitro assay was designed to produce proto-spherule like those found in AMD drusen. HAP was detected with a specific fluorescent dye. This assay was used to discover inhibitors.

The inhibition of the HX domain of Vn prevents the formation of plaques associated with age-related macular degeneration. Suitable inhibitors of the HX domain of Vn can be identified by screens (the in vitro assay).

The compounds identified in the screens will demonstrate the ability to inhibit the activity of the Vn in a human eye. These compounds include organic molecular having a molecular weight of less than 1,000 Da.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes the IC50 (the dose where 50% of the cells show the desired effects) as determined in cell culture. Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD50) (the dose lethal to 50% of the population) and the ED50) (the dose therapeutically effective in 50% of the population). The dose ratio between toxic and therapeutic effects is the therapeutic index and it can be expressed as the ratio between LD50 and ED50. Compounds which exhibit high therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. Dosage amount and interval may be adjusted individually to provide plasma levels of the active moiety which are sufficient to maintain the desired effects.

The amount of composition administered will, of course, be dependent on the subject being treated, on the subject's weight, the severity of the affliction, the manner of administration and the judgment of the prescribing physician.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

### Experiments:

### Protein Preparation

Vn was prepared from Escherichia coli. All buffer solutions were prepared with Milli-Q deionized water. For calcium-free preparations, the protein was folded by dropwise dilution from buffer T1 (20 mM Tris HCl pH 8, 6 M guanidine, 10 mM dithiothreitol) into buffer T2 (20 mM Tris HCl pH 8, 500 mM ArgCl, 300 mM NaCl, 5 mM β-mercaptoethanol, 1 mM hydroxyethyldisulfide), followed by dialysis into buffer MI (20 mM MES, pH 6.5, 300 mM NaCl) and size exclusion chromatography (Superdex 200 10/300 GL, GE Healthcare). Calcium-containing samples were prepared by supplementing buffer MI with CaCl₂).

### Compound Identification and Optimization

### In Vitro Drusen Model: Production of HAP-Protein-Lipid Proto-Spherules

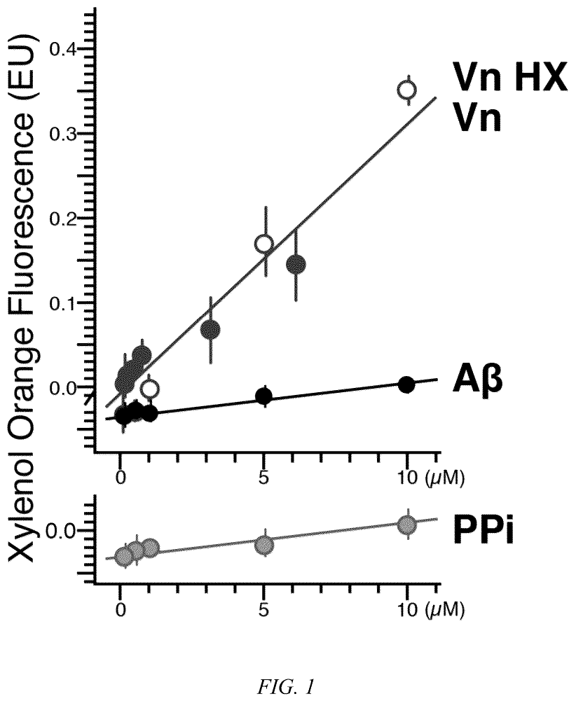

In this model, fluorescence emission reflected the amount of HAP deposited on spherules. The results are show in FIG. 1. Vitronectin increases HAP deposition on spherules in a dose-dependent and time-dependent manner. Amyloid-β (Aβ) has no significant effect on HAP mineralization. Pyrophosphate (PPi) is a mineralization inhibitor and serves as negative control. There are many HAP-binding proteins but Vn is unique: it plays an active role in HAP mineralization.

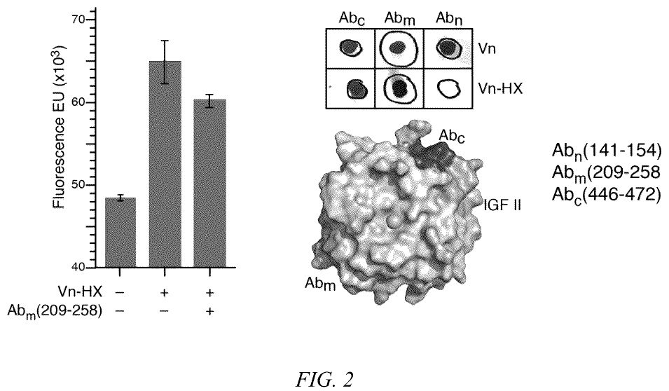

This assay was used to test Vn antibodies (need details on Ab_{c}, Abₘ and Abₙ and IGF II) as potential inhibitors. The results are shown in FIG. 2. Vitronectin promotes HAP deposition in a dose- and time-dependent manner. Antibody Abₘ inhibits Vn-dependent HAP deposition at 1:10 Abm:Vn molar ratio. The results indicate that nhibiting Vn can inhibit HAP deposition and drusen formation.

The compounds are identified by screening their inhibitory activities of Vn.

Identified compounds are further optimized by rational design.

### Inhibitory Activities

The inhibitory activities of the optimized compounds are measured.

### Ophthalmic Compositions

Ophthalmic compositions containing an effective amount of the optimized compounds are prepared and tested.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

1. A method for inhibiting activity of a human blood protein vitronectin, comprising:
administering a composition that inhibits activity of a calcium and hydroxyapatite binding site of the human blood protein vitronectin,
wherein the composition is an ophthalmic composition.

2. The method of claim 1, wherein the human blood protein vitronectin is presented in a human eye.

3. The method of claim 1, wherein the composition comprises an effective amount of an organic compound having a molecular weight of less than 1,000 Daltons.

4. (canceled)

5. The method of claim 1, wherein the composition further comprises one or more selected from the group consisting of a thickening agent, a pH adjustor, a wetting agent, a stabilizer, a solubilize, a preservative, a refreshing agent, and an ointment base.

6. The method of claim 5, wherein the thickening agent is gellan gum or xanthan gum.

7. The method of claim 3, wherein the calcium and hydroxyapatite binding site is an HX domain of the human blood protein vitronectin.

8. The method of claim 7, wherein the organic compound binds to the HX domain.

9. The method of claim 1, wherein the method is for treating geographic atrophy or age-related macular degeneration.

10. An ophthalmic composition comprising:
an effective amount of an organic compound that inhibits the binding of HAP/Ca to Vn and having a molecular weight of less than 1,000 Daltons; and
one or more selected from the group consisting of a thickening agent, a pH adjustor, a wetting agent, a stabilizer, a solubilize, a preservative, a refreshing agent, and an ointment base.

11. The ophthalmic composition of claim 10, wherein the thickening agent is gellan gum or xanthan gum.

12. The ophthalmic composition of claim 10, wherein the pH adjustor is selected from the group consisting of hydrochloric acid, citric acid, phosphoric acid, acetic acid, sodium hydroxide, potassium hydroxide, boric acid, borax, sodium carbonate, sodium hydrogencarbonate and tris(hydroxymethyl)aminomethane.

13. The ophthalmic composition of claim 10, wherein the wetting agent is selected from the group consisting of glycerin, carboxymethylcellulose, hydroxypropyl methylcellulose, mannitol, polyvinyl alcohol (PVA), and hydroxyethylcellulose.

14. The ophthalmic composition of claim 10, wherein the ophthalmic composition is an ophthalmic solution or an ophthalmic ointment.

15. The ophthalmic composition of claim 10, wherein the ophthalmic composition is for treating geographic atrophy or age-related macular degeneration.

16. (canceled)

17. (canceled)

18. (canceled)

19. (canceled)

20. (canceled)

21. (canceled)

22. (canceled)

23. (canceled)

24. An ophthalmic composition comprising:
an effective amount of an organic compound that inhibits vitronectin-dependent hydroxyapatite deposition and having a molecular weight of less than 1,000 Daltons; and
one or more selected from the group consisting of a thickening agent, a pH adjustor, a wetting agent, a stabilizer, a solubilize, a preservative, a refreshing agent, and an ointment base.

25. The ophthalmic composition of claim 24, wherein the thickening agent is gellan gum or xanthan gum.

26. The ophthalmic composition of claim 24, wherein the pH adjustor is selected from the group consisting of hydrochloric acid, citric acid, phosphoric acid, acetic acid, sodium hydroxide, potassium hydroxide, boric acid, borax, sodium carbonate, sodium hydrogencarbonate and tris(hydroxymethyl)aminomethane.

27. The ophthalmic composition of claim 24, wherein the wetting agent is selected from the group consisting of glycerin, carboxymethylcellulose, hydroxypropyl methylcellulose, mannitol, polyvinyl alcohol (PVA), and hydroxyethylcellulose.

28. The ophthalmic composition of claim 24, wherein the ophthalmic composition is an ophthalmic solution or an ophthalmic ointment.

29. The ophthalmic composition of claim 24, wherein the ophthalmic composition is for treating geographic atrophy or age-related macular degeneration.

30. (canceled)

31. (canceled)

32. (canceled)

33. (canceled)
